(19) United States
(12) Patent Application Publication
(10) Pub. No.: US 2008/0206752 A1
(43) Pub. Date: Aug. 28, 2008

(54) Method For the Photochemical Attachment of Biomolecules to a Substrate

(75) Inventors: Maxime Balakirev, Grenoble (FR); Jan Sudor, Toulouse (FR); Francois Chatelain, Beaulieu (FR); Xavier Coqueret, Phalempin (FR)

Correspondence Address: OBLON, SPIVAK, MCCLELLAND MAIER & NEUSTADT, P.C., 1940 DUKE STREET, ALEXANDRIA, VA, 22314, US

(73) Assignees: COMMISSARIAT A L'ENERGIE ATOMIDQUE, Paris (FR); AbAg, Chilly-Mazarin (FR)

(21) Appl. No.: 11/815,935

(22) PCT Filed: Feb. 10, 2005
(86) PCT No.: PCT/EP2005/002366

§ 371 Date: Feb. 21, 2008

(51) Int. Cl. C12Q 1/68; C12N 9/96

(52) U.S. Cl. 435/6; 435/188

(57) ABSTRACT

Methods and devices for attaching biomolecules to a solid substrate surface for example to the inner surface of a capillary. In particular, the invention relates to compounds and methods for creating patterned arrays of biomolecules inside fused silica capillaries so that a plurality of bioassays can be conducted simultaneously.

The invention is directed to a method for the photochemical attachment of biomolecules to a substrate, and to a device permitting such a method to be operated.

The present invention relates to methods and devices for attaching biomolecules to a solid substrate surface for example to the inner surface of a capillary. In particular, the invention relates to compounds and methods for creating patterned arrays of biomolecules inside fused silica capillaries so that a plurality of bioassays can be conducted simultaneously.

The immobilization of biological molecules on surfaces is a critical step in many bioassays including diagnostic analysis, high-throughput screening, and bioelectronic sensing [Zammatteo, N. et al., Biotechnol. Annu. Rev. 2002, 8, 85-101; Russo, G. et al., Oncogene 2003, 22, 6497-6507; Ratner, D. M. et al., Chembiochem 2004, 5, 379-382; Reimer, U. et al., Curn. Opin. Biotechnol. 2002, 13, 315-320; Xiao, Y. et al., Science 2003, 299, 1877-1881; Yeo, W. S. et al., Angew. Chem. Int. Ed. Engl. 2003, 42, 3121-3124]. Most current bioassay techniques allow for the detection of only one individual sample analyte per experiment. Although sequential detection and identification strategies are possible, the ability to detect and identify multiple analytes simultaneously within a single sample may be highly advantageous. For example, the analysis of environmental samples could be performed faster, cheaper, and with less test-to-test variability if the analyses for multiple analytes could be performed concurrently rather than sequentially. Transcriptome, proteome analysis as well as drug screening provides another area where one is usually interested in detecting and identifying more than one possible analyte. Finally, diagnostic analysis often requires the measurement of several related factors to accurately diagnose the pathology.

One of the best approaches to multi-analyte detection and identification is to use spatial isolation of detectable elements on a solid support, i.e. molecule array. In this approach, analytes are detected and identified not by which label is detected and identified, but rather by where on the substrate the label is positioned on array. Prior knowledge of the type of biomolecule or bioactive agent immobilized on the discrete region of the array allows for the identification and quantitation of multiple analytes. For molecule arrays a plane format is typically employed where the positional control (i.e. micropatterning) is achieved through robotic or photochemical addressing of the molecules and their subsequent attachment to the surface [Fodor, S. P. et al., Science 1991, 251, 767-773; MacBeath, G. et al., Science 2000, 289, 1760-1763; Okamoto T. et al., Nat. Biotechnol. 2000 April; 18(4):438-41; Lange S A. et al., Anal Chem. 2004 Mar. 15; 76(6):1641-7].

Microfluidic capillary format offers several advantages relative to plane format, which include a much smaller sample volume, high surface-to-volume ratio, kinetically rapid reactions at the surface, the potential for automated fluid delivery and analysis of many samples in parallel [Delamarche E. et al., Science. 1997 May 2; 276(5313):779-81; Sia S K. et al., Angew Chem Int Ed Engl. 2004 Jan. 16; 43(4):498-502; Zhan W. et al., Anal Chem. 2002 Sep. 15; 74(18):4647-52; Cousino M A. et al., Anal Chem. 1997 Sep. 1; 69(17):544A-549A]. The fused silica capillaries are widely used in electrophoretic [Horvath J, et al., Electrophoresis. 2001; 22(4):644-55] and flow-through analytical systems [Holt D B. et al., Anal Biochem. 2000 Dec. 15; 287(2):234-42; Narang U. et al., Anal Biochem. 1998 Jan. 1; 255(1):13-9; Koch S. et al., Biosens Bioelectron. 2000 January; 14(10-11):779-84], though a closed geometry of the capillary limits significantly the use of conventional immobilization methods for molecule arraying. To our knowledge the only method reported to date is a coating of the capillary with a photosensible 2-nitro-5-[11-(trimethoxysilyl)undecyl]oxybenzyl methoxy poly-(ethylene glycol) propanoate (NMPEG-silane). The authors described a pattern of surface bound aldehyde groups generated by irradiation of NMPEG-silane that can be used for protein immobilization via Schiff base (U.S. Pat. No. 5,773,308). Other successful strategies to circumvent the problem of arraying inside capillary include: (1) the patterning inside plastic capillaries based on a microsyringe injection and physical adsorption of the proteins [Misiakos K. et al., Biosens Bioelectron. 1998 Oct. 1; 13(7-8):825-30; Petrou P S. et al., Biosens Bioelectron. 2002 April; 17(4):261-8]; (2) the fabrication of elastomeric microfluidic channels that surround the molecule arrays patterned on the plane surface [Delamarche E. et al., Science. 1997 May 2; 276(5313):779-81; Sia S K. et al., Angew Chem Int Ed Engl. 2004 Jan. 16; 43(4):498-502; Zhan W. et al., Anal Chem. 2002 Sep. 15; 74(18):4647-52]; and (3) the incorporation of molecule-bearing microbeads inside microchannels [Sato K. et al., Anal Chem. 2001 Mar. 15; 73(6):1213-8; Noda H. et al., Anal Chem. 2003 Jul. 1; 75(13):3250-5].

U.S. Pat. No. 5,482,867 describes a method of immobilizing anti-ligands on a surface of a substrate by attaching to the substrate a caged biotin analog that has a photolabile protecting group. The protecting group is removeable by irradiation to convert the caged biotin analog into a biotin analog that is capable of non-covalently immobilizing an anti-ligand. Sequential steps of masking, irradiation and immobilization may be carried out to create a patterned substrate having different anti-ligand bound to different regions. Similar methods are described in U.S. Pat. No. 5,412,087, U.S. Pat. No. 5,391,463, U.S. Pat. No. 5,451,683, U.S. Pat. No. 5,489,678, U.S. Pat. No. 4,562,157, U.S. Pat. No. 5,316,784, U.S. Pat. No. 5,252,743 and U.S. Pat. No. 5,143,854.

J. H. McAlear et al., U.S. Pat. No. 4,103,064 and U.S. Pat. No. 4,103,073 disclose the attachment of thick films of proteins and subsequent microlithographic patterning using standard resist technology. The drawbacks of this method include: (1) many steps are involved; (2) there is no covalent attachment between the protein and the substrate; (3) many resists entail the use of organic solvents such as diglyme that are known to denature proteins; (4) the development of many resists require the use of alkaline developers which may denature proteins; (5) the crosslinking agent glutaraldehyde is known to denature proteins.

US20030378440 discloses an apparatus for detecting nucleic acid molecules such as target DNA molecules and mRNA molecules by using a DNA probe, and provides a DNA capillary, comprising a fluid passageway formed of a cyclindrical capillary made of glass, a plurality of independent probe regions formed in the inner wall of the fluid passageway, and DNA probes each immobilized in the probe region, the immobilized DNA probes differing from each other. For performing the measurement, a sample is introduced through an open portion into the capillary so as to perform reaction and, then, fluorimetry.

Photo-irradiation has been used frequently to graft polymers onto a surface, as is illustrated in the following references: M. Ulbricht et al., Journal of Membrane Science, 115, 1996, 31-47; W. Yang et al., J. Applied Polymer Science, vol. 62, 533-543, 1996; W. Yang et al., J. Appl. Polym Science, vol. 162, 545-555 (1996); G. Geushens et al., European Polymer Journal, 36, 2000, 265-271.

Several documents disclose a solid substrate grafted with molecules comprising a function which is protected by a photo-removable protective group. Selective photo-irradiation permits the liberation of the function. A coupling reaction is then performed between the free function and a biomolecule. With this two-steps method patterned grafting of a substrate with different biomolecules can be obtained (WO 98/34913, US20030148367). However, the necessity to operate in two steps makes these methods complicated and tedious.

Another way of grafting a substrate with a biomolecule consists in polymerizing, by radiation grafting, monomers onto a substrate, wherein some of the monomers are conjugated or covalently bonded to a biomolecule. Such a method is disclosed in U.S. Pat. No. 5,034,428 and U.S. Pat. No. 5,453,461. However this method does not permit patterned grafting of a substrate.

Document U.S. Pat. No. 4,562,157 discloses a device useful for diagnostic. Two or more biomolecules are attached to a sensor. This device is made by the following method: a group having a photo-activatable function is covalently bonded to the sensor's surface. The modified surface is photo-exposed through a mask and the biochemical species in solution is selectively bonded to the irradiated zone. This method allows the production of printed circuits for proteins. Photo-activation techniques permit precise selection of very small areas. Supports are slices of silica grafted with N-(4-azido-2-nitrophenyl)-1,3-diaminopropanes as a photo-activatable group. The biomolecule is linked to the silica support by the intermediary of the photo-activatable compound. However, the method disclosed in this document is associated with several disadvantages:

The photo-activator is an azido functionalized molecule. Such photo-activators have the disadvantage of decomposing after they have been irradiated, whereas carbonyl photo-activators, for example, can be repeatedly irradiated and remain active until they react with a target molecule.

Selective irradiation-dependent grafting of biomolecules is theoretically possible if no non-selective attachment of biomolecules to the surface occurs. However, when put into practice such non selective grafting is observed when grafting is directly operated on the solid substrate in the presence of a photo-initiator, as is taught in this document.

Lowe et al., U.S. Pat. No. 4,562,157, describe patterns of covalently attached biomolecules deposited on photo-activated portions of a silane film. However, the disclosed method does not overcome the problem of non-specific attachment of biomolecules to unmodified portions of the silane film.

It has been an aim of the present invention to operate patterned grafting of a biomolecule on a solid substrate, in conditions such that non specific grafting is avoided. Surprisingly, such an aim has been reached in the following conditions:

The invention is directed to a method for the grafting of a molecule to a solid substrate, wherein the solid substrate itself comprises a layer of a linker that has a resistance to the adsorption of the molecule, said method comprising the steps of:
contacting the solid substrate with a solution wherein the molecule to be grafted and a photo-sensitizer are solubilized;
photo-irradiating at least one part of the solid substrate.

The selective photo-irradiation of a selected part of the solid substrate causes the grafting of the molecule onto the solid substrate while avoiding non specific attachment on the non irradiated part of the substrate. Another object of the invention is the use of this method to obtain specific attachment of the molecule to the irradiated part of the substrate.

Surprisingly, when the photo-sensitizer is solubilized with the molecule to be grafted, the specificity of attachment is very high, and non specific attachment is avoided. On the contrary, when the photo-sensitizer is covalently bonded to the solid substrate or just adsorbed onto that solid substrate, it has been observed that non specific attachment took place.

Contrarily to what has been disclosed in some prior art documents like U.S. Pat. No. 5,773,308, grafting is the direct consequence of photo-irradiation and no further coupling step is necessary.

The invention can be used for the attachment of any organic molecule to a substrate.

The invention is particularly directed to the attachment of biomolecules, like proteins, sugars, lipids, steroids, enzymes, peptides, glycoproteins, nucleic acids (RNA, DNA) and their analogs. The latter include artificially designed chemical molecules that resemble DNA, having the modified bases (other than A, T, U, C, G), the sugar (other than ribose or deoxyribose), or the backbone (other than a phosphodiester bond).

The invention is especially concerned by the attachment of biomolecules mentioned above to substrates with biomolecule-repellent properties. Such substrates are obtained by the choice of an appropriate layer of a linker. Often, biomolecules are hydrophobic molecules and the substrate is of hydrophilic nature.

The proposed method for photochemical immobilization of molecules can be applied for different kinds of substrates including glass, silicon, fused silica, polymers, metals, metal oxides and ceramics. The substrate is not limited to planar surfaces but can also be selected among beads, microtiter plates and particularly capillaries.

Microfluidic capillary format offers several advantages relative to plane format, which include a much smaller sample volume, high surface-to-volume ratio, kinetically rapid reactions at the surface, the potential for automated fluid delivery and analysis of many samples in parallel. The capillaries which can be used as solid support for the attachment of biomolecules include glass, silicon, fused silica, plastic (polymer) capillaries etc. They can have varied geometry such as cylindrical or rectangular shape, 180° closed or open from one side (for example micromachined silica channels or plastic molds). One of the examples is the fused silica capillaries, which are widely used in electrophoretic and flow-through analytical systems. The closed geometry of this capillary limited significantly the use of conventional techniques for molecule immobilization.

The proposed method of photochemical attachment of molecules allows the standard flexible fused silica capillaries with UV transparent coating to be used for molecule immobilization. These capillaries are robust enough to be easily manipulated and to be used in different kinds of chemical modification. Moreover the chemistry of the modification of inner fused silica surface, that can include, for example the application of silanes or/and polymer grafting, is well developed. The use of UV transparent coating allows the irradiation of the inner surface to be performed through the capillary wall. This coating also allows the detection of the fluorescent species inside the capillary, which is an important step for the development of analytical systems and in-capillary arrays.

The capillaries are available from commercial sources such as Polymicro Technologies or FiberTech GmbH, which provide: (a) Wide ranges of sizes with internal diameters less than 1 micron up to several millimetres and outer diameters down to 60 microns and up to several millimetres; (b) Various internal and external geometries; (c) Excellent chemical durability and robustness; (d) Silica and quartz that are stronger than steel; (e) Materials that are easy to cleave or cut; (g) Tight yet economical tolerances etc.

One of the most important advantages of the capillaries is their small volume: for example, the capillaries with an outer diameter of 363 μm and an inner diameter of 100 μm have a filling capacity of about 78.5 nl per cm of the capillary. Due to their closed geometry there is no problem of evaporation even with nanoliter sample volumes. Moreover the experimental conditions (temperature, concentration, etc.) can easily be controlled inside the capillaries thus making possible a wide variety of bioassays. Another advantage of the capillaries is that they can be integrated into an analytical lab-on-a-chip microsystem that combines sample preparation, analysis, and data treatment. Chemical modification of the capillary as well as immobilization, patterning, and treatment of biomolecules and data acquisition can also be automated.

The solid substrate itself comprises a layer of a linker, a component that has a resistance to the adsorption of biomolecules. It can be the synthetic hydrophilic poly(oligo)mer (poly(oligo)ethylene glycol (PEG) and polyacrylamide (PA) derivatives, etc.) or natural poly(oligo)mer (proteins, poly(oligo)-carbohydrates).

Favourite polymers to be used as a linker with biomolecule repellent properties are polymers containing amino or H-donor groups that can be easily activated by photo-sensitizers. Such polymers and their grafting onto a solid substrate will be described hereunder:

In the present invention, the repellent linker is preferably based on polymer brushes [FIG. 8B], preferably dense, hydrophilic and exhibiting antifouling properties, i.e., they prevent non-specific adsorption of biomolecules onto such surfaces.

Theoretically, two limiting regimes exist in connection with polymer grafting density [Alexander, S., J. Phys. (Paris), 1977, 38, 983; De Gennes, P. G., Macromolecules, 1980, 13, 1069]: at low grafting densities, each chain is isolated occupying roughly a half-sphere with a radius comparable to the radius of gyration (“mushroom” regime, FIG. 8 A); at high grafting densities, polymers stretch away from the interface to avoid overlapping, forming a polymer brush [FIG. 8 B] [Milner, S. T., Science, 1991, 251, 905].

Additionally, end-grafted polymers used to build the linker layer contain amino or H-donor groups that can easily be activated by photoinitiators, benzophenone, anthraquinones, thioxanthones, benzyls, etc., dissolved freely in a solution, upon irradiation by a light (e.g., UV light of 365 nm). The activated polymer brushes contain many free radicals that can easily and rapidly react with molecules of interest dissolved in a solution, and attach them covalently to the surface. It should be noted that the photo-initiators may also activate molecules dissolved in a solution, however, the activation happens preferably on the a-carbon situated next to the amino or H-donor groups of the polymer. In both cases, however, upon activation of the surface grafted polymers or the free biomolecules, covalent bounds will be formed between the polymer and the biomolecules. The surface-grafted polymers play the role of a linker between the solid support and the immobilized molecules.

The polymer brushes are synthesized preferably from acrylamide or its derivatives that carry amino or H-donor groups, such as N-(3-aminopropyl) methacrylamide, N-[(3-dimethylamino) propyl]methacrylamide, and N-[tris-(hydroxymethyl)-methyl]acrylamide. However, other polymers carrying H-donor or amino groups, such as polyethylene glycols (PEG), polyethylene oxides (PEO), copolymers of PEG with polypropylene glycols (PPG), copolymers of PEO with polypropylene glycols (PPG), copolymers of PEG with polypropylene oxides (PPO), copolymers of PEO with polypropylene oxides (PPO), copolymers of PEG with polydimethylsiloxanes (PDMS), copolymers of PEO with polydimethylsiloxanes (PDMS), copolymers of PEG with derivatives of polyacrylamides, and copolymers of PEO with derivatives of polyacrylamides may be used for the surface modifications.

The polymer brushes can be prepared according to the methods described in the literature [Hjerten, S., J. Chromatogr., 1985, 347, 191-198; Cobb, K. A. et al, Anal. Chem., 1990, 62, 2478-2483; Herren, B. J. et al., J. Colloid Interface Sci., 1987, 115, 46; Balachander, N. et al., Langmuir, 1990, 6, 1621; Burns, N. L. et al., Langmuir, 1995, 11, 2768; Fouassier, J.-P., Photoinitiation, Photopolymerization, and Photocuring: Fundamentals and Applications, Carl Hanser Verlahg, Munich Vienna New York, 1995; Ulbrich, M. et al., J. Membrane Sci., 1996, 115, 31-47; Yang, W. et al., J. Appl. Pol. Sci., 1996, 62, 533-543; Yang, W. et al., J. Appl. Pol. Sci., 1996, 62, 545-555; Geuskens, G. et al., European Polymer Journal, 2000, 36, 265-271]. In a first approach, a polymer chain is polymerized starting from a modified solid substrate (such as, glass, quartz, silica, silicon, PDMS, or a plastic material, such as polycarbonate, polypropylene, polyethylene, polyamides, polysulfone, etc.) [Hjerten, S., J. Chromatogr., 1985, 347, 191-198; Cobb, K. A. et al., Anal. Chem., 1990, 62, 2478-2483]. Secondly, a pre-synthesized polymer chain can be grafted onto a solid substrate [Herren, B. J. et al., J. Colloid Interface Sci., 1987, 115, 46; Balachander, N. et al., Langmuir, 1990, 6, 1621; Burns, N. L. et al., Langmuir, 1995, 11, 2768].

The photo-sensitizers (PS) are compounds which are stable in ambient light and can be activated by photo-irradiation. Preferentially they are selected among carbonyl-containing photo-sensitizers. For example, one can use water soluble benzophenone, anthraquinone, camphorquinone, thioxanthone derivatives, benzophenone iso(thio)cyanate, substituted benzoylbenzoic acids, benzoylbenzylbromide, (benzoylbenzyl)-trimethylamimonium chloride, anthraquinone, anthraquinone-containing carboxylic acids, bromomethyl-anthraquinone, anthraquinone sulfonic acid, camphorquinone-10-sulfonic acid etc.

The carbonyl-containing photo-sensitizers were chosen because of their remarkable chemical and photochemical robustness; these molecules are more stable in ambient light than other known photo-active compounds, and can be repeatedly activated at 330-365 nm (400-500 for camphorquinone) in aqueous solution without significant loss of activity. Moreover, their chemistry is well known and various derivatives can be easily synthesized.

The major exited state of benzophenone, a carbonyl centered n,π*di-radicaloid triplet, is able to abstract the hydrogen atom from organic residues and to cross-link with resulting radicals, the main sum reaction being the insertion into the C—H bond. Usually high yields of photo-cross-linking (up to 80%) are obtained.

Excited anthraquinone has also the triplet n,π*state as the lowest excited state and is a powerful electron acceptor. The presence of a hydrogen donor leads to rapid quinine reduction either by a one step hydrogen atom abstraction or by a two step electron/proton transfer. In both cases a radical pair is generated on the quinine and on the hydrogen donor (e.g., biomolecule). The relative rate in hydrogen atom abstractions of excited anthraquinone is very high, and, as a result, the excited anthraquinone radicalizes almost any exposing C—H group.

Camphorquinone strongly absorbs light in the UV (π,π*transition) and exhibits a slight absorption at 470 nm with a low adsorption coefficient indicating a n,π* transition. This latter absorption band gives to camphorquinone the possibility to be used as a visible photo-sensitizer.

According to the invention, a water-soluble PS is co-dissolved in a solution of a molecule (preferably a biomolecule) in water or in an organic solvent. The irradiation of this solution results in PS-dependent photo-sensitization and in the generation of reactive species from the biomolecules and from the solid support's surface, which finally results in the formation of a covalent linkage between the biomolecules and the solid support. The most important advantage of this method is that it makes possible the use of different surface coatings which are resistant to on-specific biomolecule adsorption such as synthetic hydrophilic poly(oligo)mer (PEG and PA derivatives etc.) or natural poly(oligo)mer (proteins, poly(oligo)-carbohydrates). This method is illustrated by FIG. 1.

According to a variant of the invention, a PS is physically associated with a biomolecule to be grafted to the solid support. The attachment can be covalent as well as non-covalent.

(a) In the case of non-covalent immobilization, a PS is attached to the biomolecule via hydrophobic, electrostatic, or affinity interactions. The affinity interactions include all kinds of ligand-protein interaction (for proteins), complementary interactions (for nucleic acids and their analogs) or others (for example, his-tag-Ni-nitrilo-triacetic acid (NTA), or biotin-streptavidin).

(b) In the case of covalent binding of the photo sensitizer to the biomolecule, specific chemical reactions are used. The electrophile-nucleophile reactions are the reactions of choice, but others such as different kinds of molecular addition, disulfide bond formation, polymerization etc. . . . can also be used. For proteins, some amino acids can be replaced/introduced/modified in order to confine PS labelling to specific sites on the protein.

An improvement of the photo-sensitization activity of the photo-sensitizer may be achieved by introduction of additional components such as tertiary amines, which allows stabilization and propagation of the radical species. The activity of the photo-sensitizer in this case is based on the radical production from the interaction of the photo-sensitizer, for example carbonyl-containing photo-sensitizer in the excited triplet state, with tertiary amines, which proceeds through a charge transfer intermediate (normally a triplet exciplex). Due to the charge transfer characteristics of the intermediate, its decomposition steps are highly dependent on the donor-acceptor capability of the PS and the co-photo-sensitizer. This additional component can be added in solution or be bound to the surface, this last configuration confining photo-chemical reactions to the irradiated surface area and providing a high yield of photo-chemical immobilization of biomolecules. Another improvement consists in using a tertiary amine solubilized in the solution of photo-sensitizer and molecule or bound to the solid substrate.

Different light sources that can be used include: mercury arc lamps, lasers, cathode ray tubes (CRT), light-emitting diodes (LED), Resonant Microcavity Anodes, photodiodes, broad wavelength lamps, and the like.

After irradiation, the solid substrate's surface is washed to remove non-covalently bound PS and, if the spatially resolved irradiation was used, the procedure can be repeated to attach another biomolecule to another location (surface patterning). A favourite method further comprises the steps of:
washing the substrate's surface;
contacting the solid substrate with a solution wherein a second molecule to be grafted and a photo-sensitizer are solubilized

and
photo-irradiating another region of the solid substrate.

The PS-dependent photo-chemical attachment of molecules, preferably biomolecules, to the substrate can be confined to a predefined region by using a mask. The modified substrate may then be washed to remove unbound biomolecules. The use of photo-lithography may allow the patterning of the biomolecules with sub-micron resolution. The process steps of masking, irradiating, and washing can be repeated on different surface regions with different biomolecules to create a patterned substrate that can be used as molecule array for detection of multiple interaction events in parallel. For detection, various physicochemical detection methods can be used in a detection step, said detection method being selected to provide a spatially resolved signal such as fluorescence, different kinds of luminescence, as well as radioactivity-based, SPR, mass spectrometry methods, etc. . . . A method for the detection of multiple interaction events of one analyte in parallel with at least two molecules, said method comprising the use of a molecule array, wherein said molecule array has been obtained by a method as described above, is another object of the invention.

In the case when the solid substrate is a capillary the irradiation can be performed on an inverted microscope equipped with a mercury arc lamp and an appropriate interference filter. The sample consists of a solution wherein the biomolecule to be grafted and the photosensitizer are dissolved. The capillary filled with the sample is mounted on the microscope stage and the patterning is performed by successive irradiation of the focus zone and lateral translation of the stage (FIG. 3a). The length of the zone was determined by a slit aperture and may vary from 1 μm to 3 mm. The light intensity at the capillary level can be controlled by using the filters thus allowing the control of the rate of photo-chemical attachment of biomolecules. The whole process can be integrated in one device and automated. Fluorescent images of the capillaries can be obtained by using a slide micro array scanner equipped with a microscope slide holder. To scan the capillaries, the metallic support of a slide format is fabricated having three grooves to hold capillaries in place (FIG. 3b).

The general procedure of the attachment of biomolecules inside fused silica capillary includes (FIG. 2):

(1) Pre-cleaning of the inner capillary surface by washing with active cleaning solution such as piranha (7:3 (v/v) of concentrated sulphuric acid and 30% hydrogen peroxide).

(2) Chemical modification of the inner capillary surface, which can involve several steps, the first being more frequently a treatment with a silane derivative. This silane generally contains two parts, one is used for binding to the silica surface (for example Si—H, Si—Cl, and Si—OR moieties); and another part is used to change the physicochemical characteristics of the inner capillary surface (for example PEG, PA for hydrophilic surface; n-alkyl, aromatic for hydrophobic surface; PS-bearing residues for photo-activable surface etc.), or to conduct subsequent modification steps (for example chemically reactive groups, nucleophilic or electrophilic residues for coupling reactions, unsatured and halogenated residues for polymerization reactions etc.). After the silane treatment and washing out of non-covalently bound silane, the silane-modified surface is stabilized by drying (can be done by heating at various temperatures, with or without vacuum). Next subsequent modification steps are performed where it is necessary. To increase the efficiency of the chemical reaction as well as to reduce the heterogeneity of the resulting modified surface, all modification steps can be performed at a constant reagent flow by using the gravity flow or a syringe pump.

(3) The capillary with the modified inner surface is used for the attachment of biomolecules. The methods and compounds described above are employed.

After the biomolecules have been attached to the inner capillary surface, the capillary is washed and is used in appropriate bioassays.

(4) The bioassays are performed by filling the capillary with the analyte sample, by allowing the interaction with the surface-bound biomolecule, and by detection of the result of this interaction.

(5) The detection may occur downstream from an assay region (flow through analytical system) or directly in the capillary (capillary fill device). For detection various optical methods can be used (light absorbance, fluorescence etc.) as well as radioactivity-based, electrochemical, capacitance methods, mass spectrometry etc.

This protocol is applicable for the photochemical attachment of any type of molecule and especially of biomolecules like oligonucleotides, proteins, sugars and lipids inside the capillary. Furthermore, the molecules immobilized in this way retain their biological activity and can be used in varied bioassays.

The grafted biological or chemical molecules either perform biochemical reactions or are screened against molecules in solutions.

As used herein, “biochemical” refers to reactions, processes, and protocols that employ at least one substrate and at least one enzyme. For example, the term “biochemical” can be used to refer to, but is not limited to protocols related to nucleic acid amplification such as the polymerase chain reaction (PCR), to genotyping such as micro-sequencing, or to the sequencing of nucleic acids. The term “biochemical” also includes other reactions catalyzed by an enzyme. As used herein, “chemical” refers to chemical reactions, processes, or protocols that, in at least one step, do not employ enzymatic catalysis. For example, the term “chemical” can be used to refer to organic or inorganic molecular syntheses or degradation reactions having at least one step which does not involve enzymatic activity.

As used herein, “biological” refers to reactions, processes, or protocols that may comprise living material. For example, the term “biological” can be used to refer to processes including, but not limited to, a single cell, a culture of single cells, a mass of adherent cells, an organism comprised of a single cell or multiple cells, or portions of tissues of organs. The term “biological” as used herein encompasses eukaryotes, including single-celled or multi-cellular organism, as well as prokaryotes, including bacteria, or viruses.

A device comprising a solid substrate as described above, comprising a layer of a linker which is a repellent to the adsorption of biomolecules and a solution comprising a solubilized photo-sensitizer, said solution being in contact with the solid substrate, is another object of the invention. Said device can be used to prepare patterned arrays of molecules, especially biomolecules, by 1) solubilizing the molecule into the solution of photo-sensitizer and 2) irradiating selected zones of the solid substrate. Said device is conceived according to the specificities disclosed above. More specifically, one or more of the following conditions are fulfilled: the substrate is selected from fused silica capillaries with UV transparent coating; the layer of a linker is selected from polymer brushes synthesized from: acrylamide, N-(3-aminopropyl) methacrylamide, N-[(3-dimethylamino) propyl]methacrylamide, N-[tris-(hydroxymethyl)-methyl]acrylamide, polyethylene glycols (PEG), polyethylene oxides (PEO), copolymers of PEG with polypropylene glycols (PPG), copolymers of PEO with polypropylene glycols (PPG), copolymers of PEG with polypropylene oxides (PPO), copolymers of PEO with polypropylene oxides (PPO), copolymers of PEG with polydimethylsiloxanes (PDMS), copolymers of PEO with polydimethylsiloxanes (PDMS), copolymers of PEG with derivatives of polyacrylamides, and copolymers of PEO with derivatives of polyacrylamides; the photo-sensitizer is selected among carbonyl-containing photo-sensitizers; a tertiary amine is preferably solubilized in the solution of photo-sensitizer or bound to the solid substrate.

A kit consisting of: 1) a device as described above and 2) a photo-irradiation source is another object of the invention.

Advantageously, said kit further comprises at least one detection means selected from: fluorescence, luminescence, radioactivity, SPR and mass spectrometry detection means.

Preferably, it further comprises an inverted microscope equipped with a mercury arc lamp and an interference filter.

Said device can be used to prepare patterned arrays of molecules, especially biomolecules, by 1) solubilizing the molecule into the solution of photo-sensitizer and 2) irradiating selected zones of the solid substrate. It can further be used to detect interactions of the grafted molecules with some analyte of interest.

### FIGURES

FIG. 1: Photochemical immobilization of biomolecules on a solid substrate by using photosensitizers (PS) in solution

FIG. 2: Photochemical immobilization of biomolecules inside a fused silica capillary

FIG. 3: Irradiation and Signal Acquisition Setups for biomolecules patterning and assay in the capillary

FIG. 4: Modification of the inner capillary surface with biomolecule-repellent moieties (PEG)

FIG. 5: Kinetics of photochemical patterning of oligonucleotides onto the inner surface of PEGS- and DHEAS-capillaries

FIG. 6: Kinetics of photochemical patterning of proteins onto the inner surface of PEGS- and DHEAS-capillaries

FIG. 7: Immunoassay in BP-capillary

FIG. 8A: Representaion of a polymer in the mushroom regime

FIG. 8B: Representaion of a polymer in the dense polymer brush configuration

FIGS. 9A, 9B, 9C: MALDI/TOF spectrum of peptide map from Cytochrome C

FIG. 10 and FIG. 11: Hybridization of Actine complementary oligonucleotides

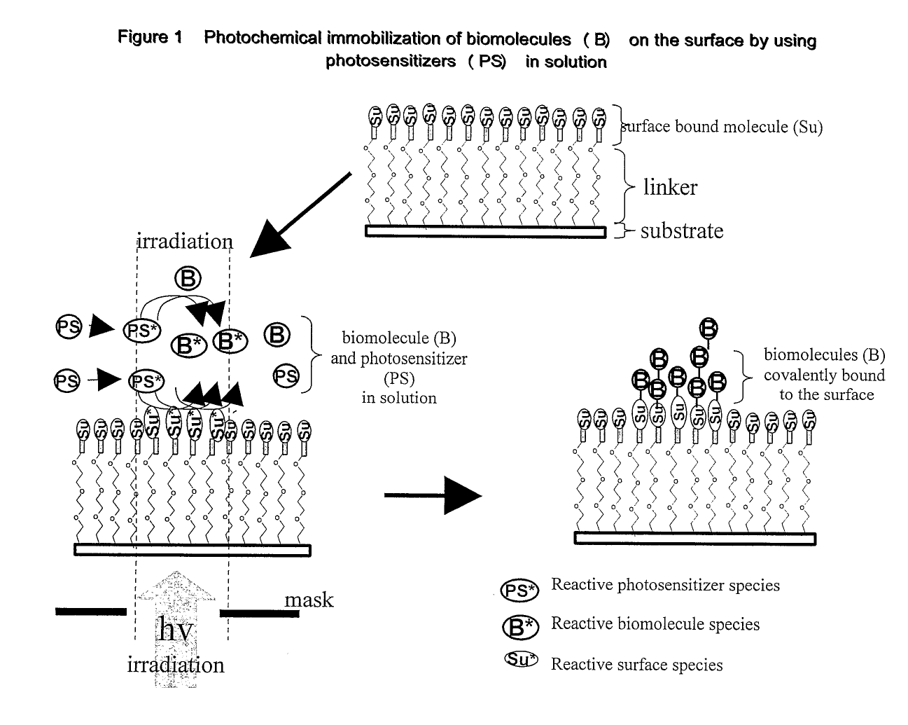

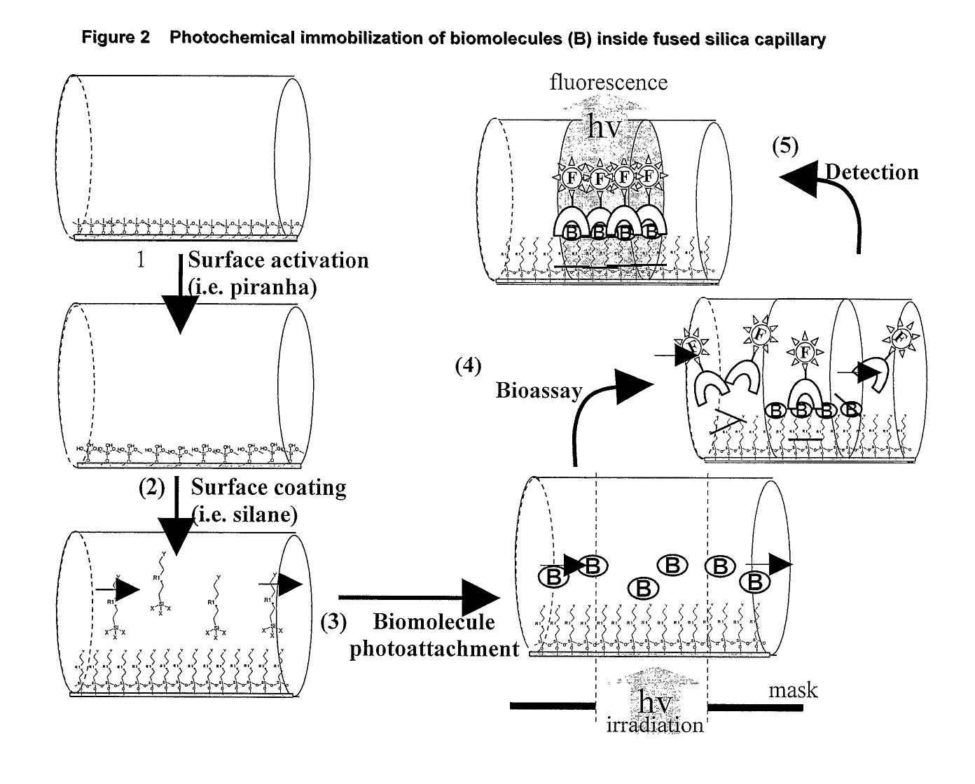

### EXAMPLES

### Example 1

### 1—Irradiation and Signal Acquisition Setups

Standard flexible fused silica capillaries with UV transparent coating are used for array construction. The capillaries have an outer diameter 363 μm and an internal diameter of 100 μm providing a filling capacity of about 314 nl per cm of the capillary. The irradiation is performed on an Olympus inverted microscope (model IX60) equipped with a 100 W mercury arc lamp and a 365 nm interference filter.

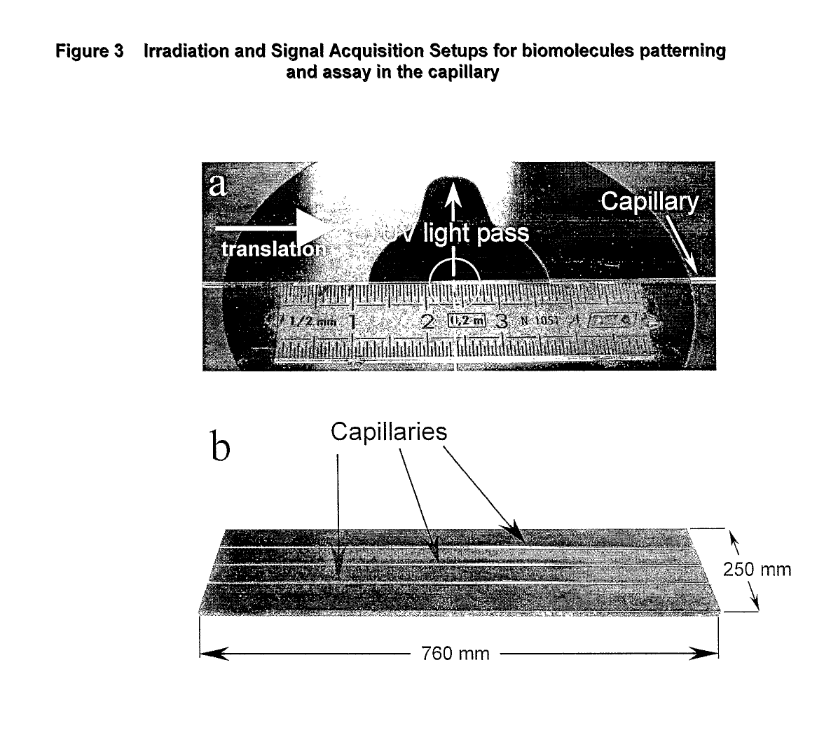

The capillary filled with sample was mounted on the microscope stage and the arraying was performed by successive irradiation of the focus zone and lateral translation of the stage (FIG. 3a). The length of the zone was determined by a slit aperture and was fixed at 160 μm. The resulting light intensity at the capillary level was measured to be approximately 2 mW/mm². Fluorescent images of the capillaries were obtained using a 4 color micro array scanner equipped with a microscope slide holder. To scan the capillaries, the metallic support of a slide format was fabricated having three grooves of 400 μm wide and 300 μm high to old the capillaries (FIG. 3b).

### 2—Coating of the Inner Surface of Fused Silica Capillary with Biomolecule-Repellent Moieties

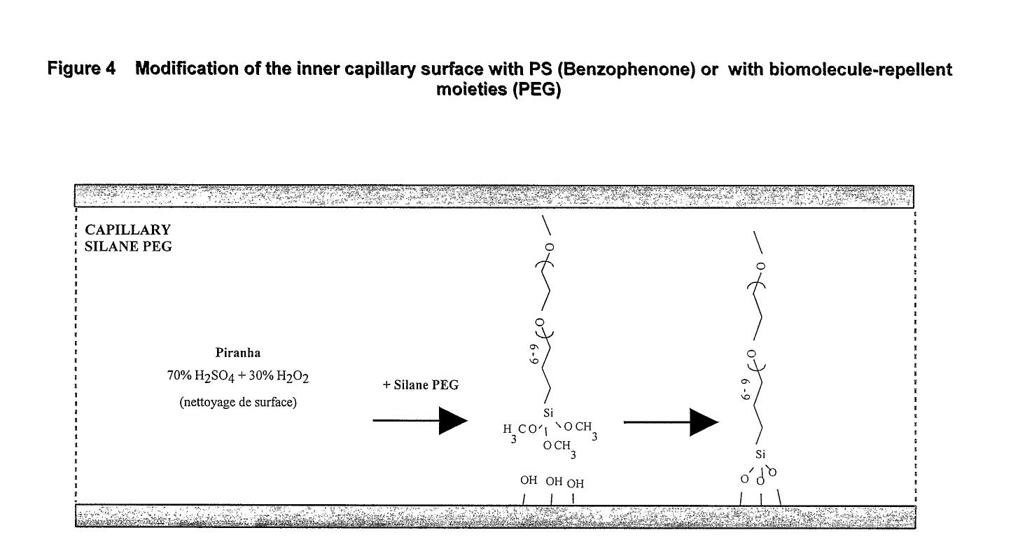

The inner surface of fused silica capillary precleaned with Piranha was aminated by using a 3% solution of PEG-silane (PEGS) or of dihydroxyethylamino-ethylsilane (DHEAS) in 95% ethanol at a constant reagent flow of 5 μl/min for 2 hours (FIG. 4). After that the capillary was flashed with ethanol (2 ml) to remove a non-covalently bound silane, dried and cured at 115° C. for 2 hours. The PEGS- and DHEAS-modified capillaries were stored at +4° C. in the dark.

### 3—Photochemical Immobilization and Patterning of Oligonucleotides Onto the Inner Surface of PEGS- and DHEAS-Capillaries

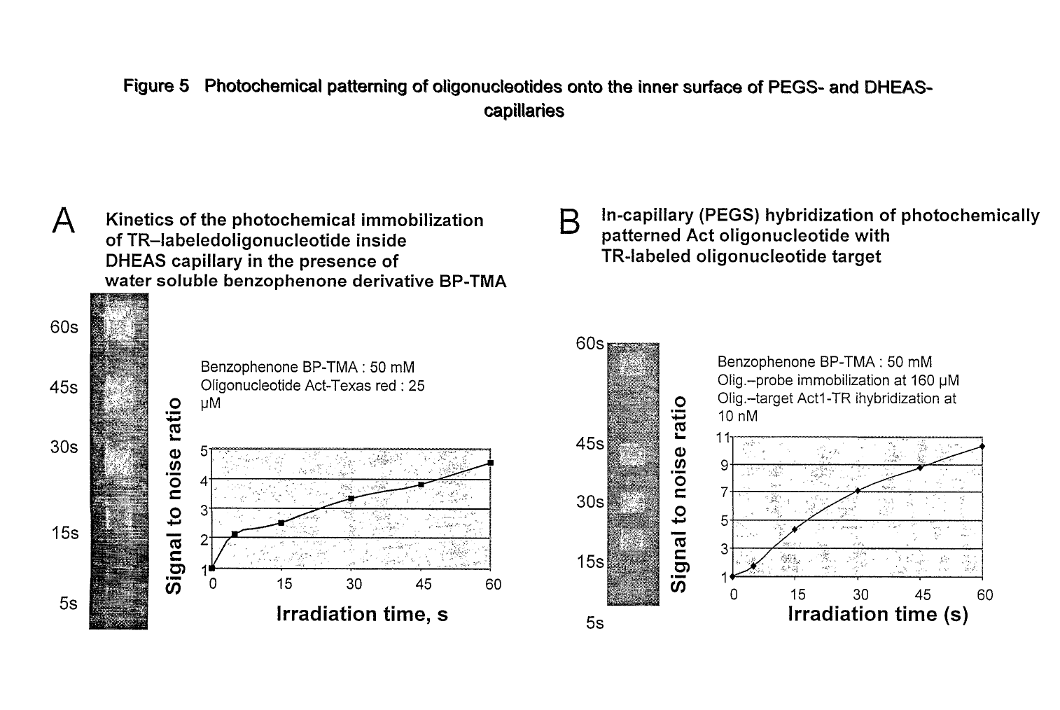

The capillaries were filled with a solution of oligonucleotide (50-600 μM) and (4-Benzoylbenzyl)trimethylammonium chloride (BP-TMA, 1-250 mM). After irradiation on the microscope setup the capillaries were washed with SSC 2× buffer and were used in further assays. This procedure resulted in the attachment of the oligonucleotide to the inner capillary surface (FIG. 5A).

### 4—Hybridization of the Oligonucleotides Immobilized on the Inner Surface of PEGS- and DHEAS-Capillaries

The hybridization of the oligonucleotides immobilized inside PEGS- and DHEAS-capillaries are shown in FIG. 5B.

### 5—Photochemical Immobilization and Patterning of Proteins onto the Inner Surface of PEGS- and DHEAS-Capillaries

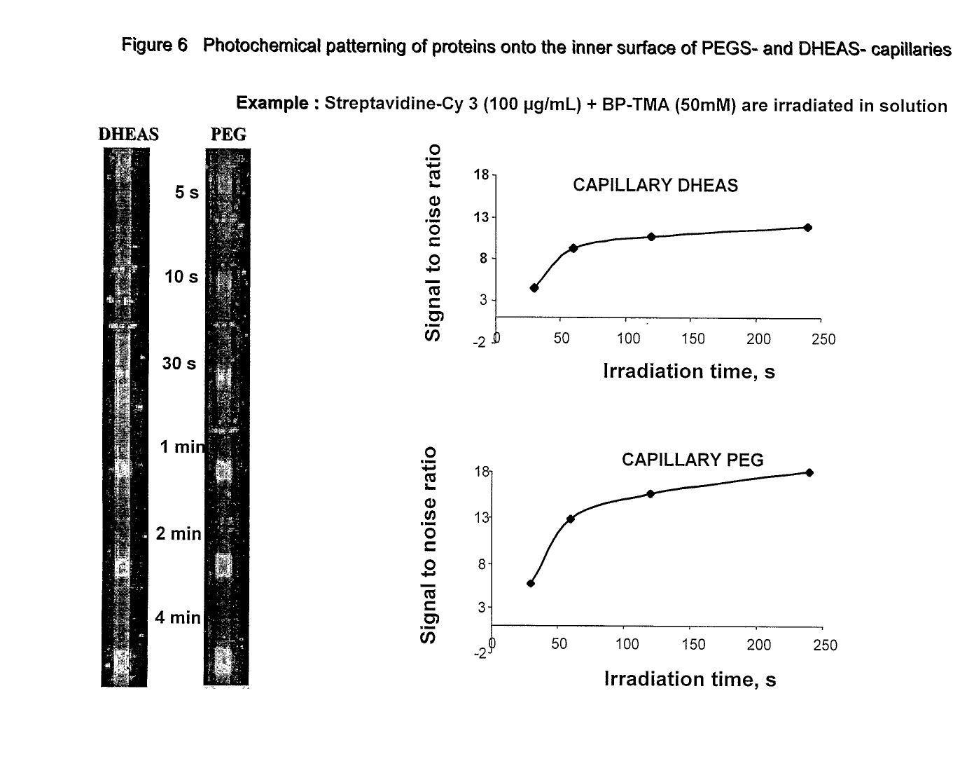

The immobilization and patterning of Cy3 and Cy5-labeled streptavidin inside PEGS- and DHEAS-capillaries are shown in FIG. 6.

### 6—Immunoassay in Capillary Format

The immunoassay in a capillary format was examined by using two different antigens: viral protein-base (PB), and bacterial listeriolysin O (LLO). The PB is a highly antigenic capsid protein implicated in cell entry of human adenovirus Ad3, an agent pathogen causing gastroenteritis and meningitis in children. The LLO is one of the major virulence factors of Listeria monocytogenes, an ubiquitous food-borne Gram-positive bacterium, responsible for septicemia and meningitis in immunocompromised persons. The recombinant proteins and their polyclonal rabbit antisera were used in immunoassay.

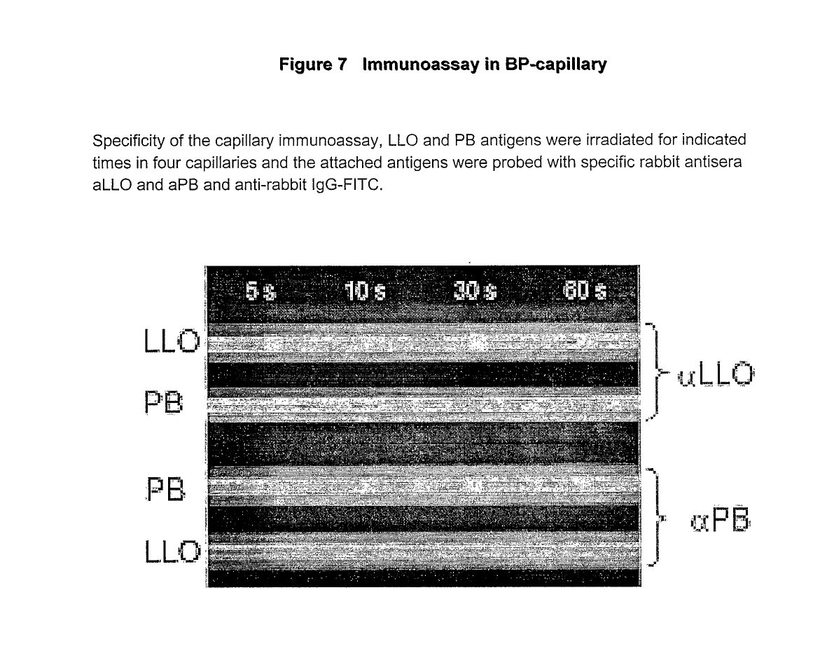

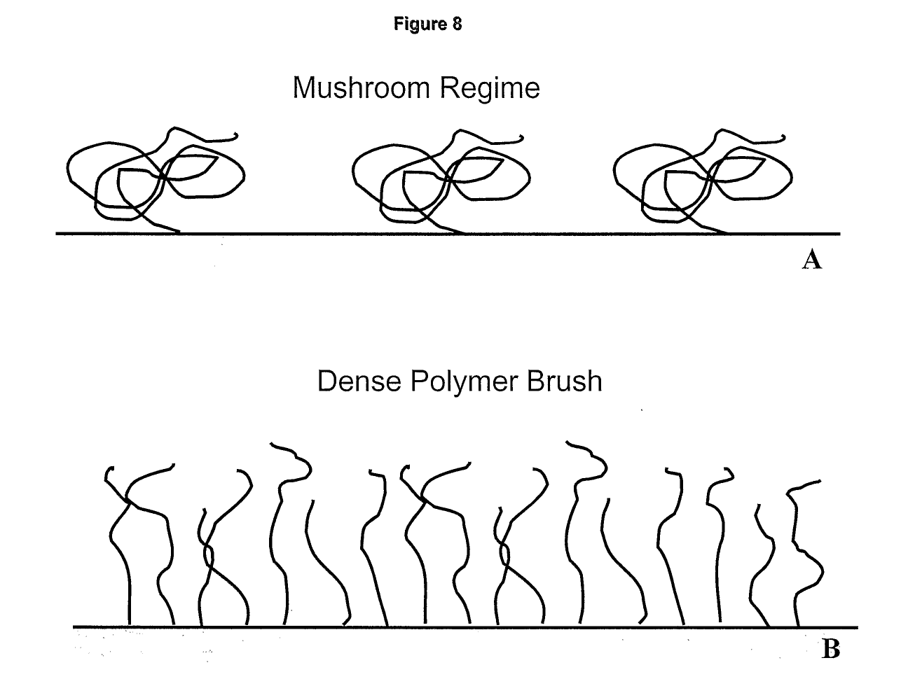

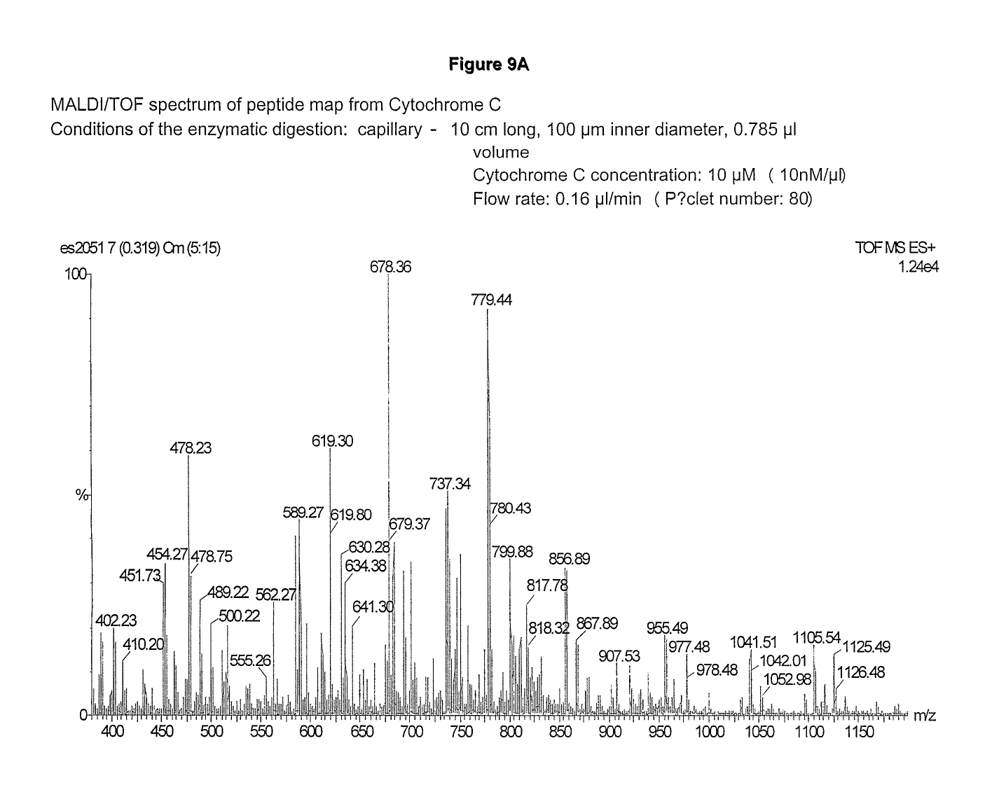

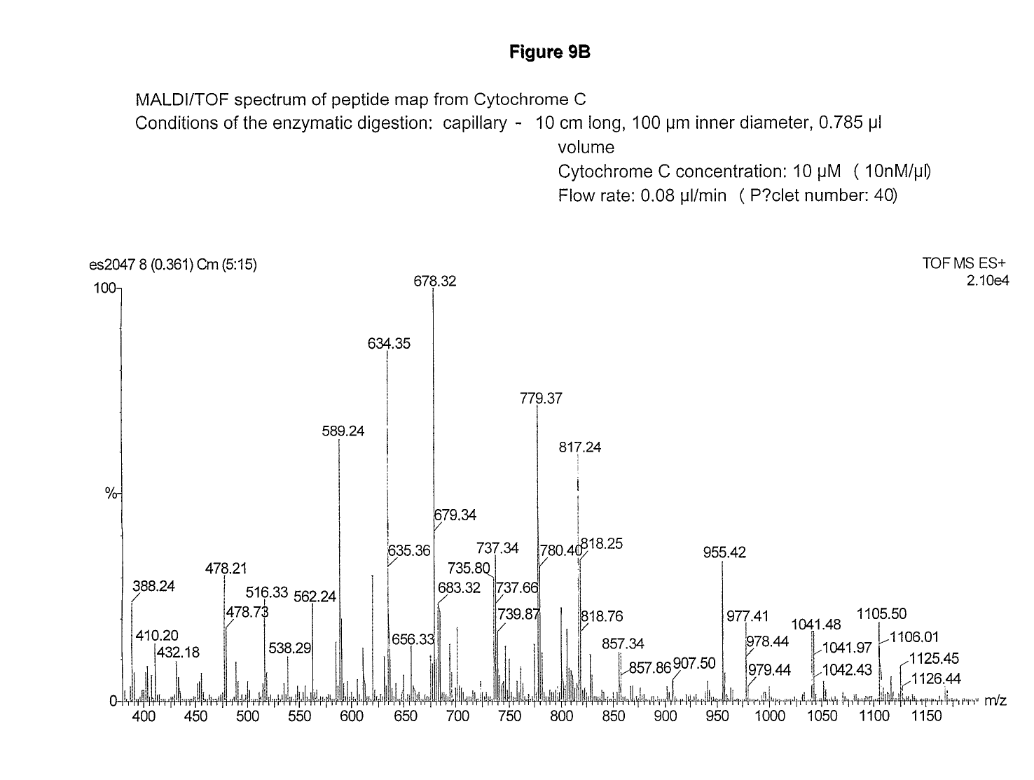

The antigens at 0.2 mg/ml were arrayed in parallel in four capillaries and resulting arrays were flushed with 1% BSA and 0.025% Tween in PBS for 15 min. Next, the capillaries were treated successively with a-PB (1:10000 dilution) or a-LLO (1:2000) antiserum in PBS/Tween, with PBS/Tween and with 5 μg/ml IgG-FITC. FIG. 7 demonstrates that the capillary array can reliably distinguish the specific antisera. The dilutions of antisera used in this experiment corresponded to that of standard ELISA assay. It should be noted that measurable signals could be obtained with higher antisera dilutions, 1:60000 for α-PB (dilution) and 1:10000 for α-LLO, demonstrating the sensitivity of the capillary array.

### Example 2

### Immobilization of Proteins (Enzymes) on Polymer Coated Surfaces

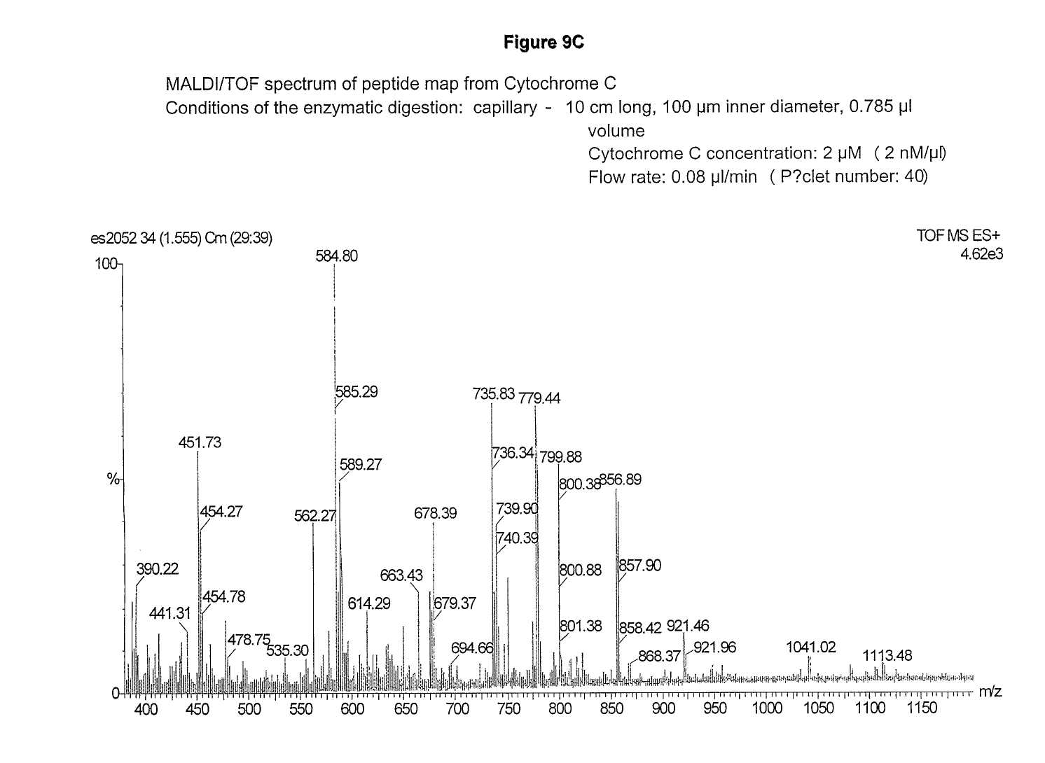

This example, with reference to FIG. 9, discusses one embodiment of the present invention and is directed to immobilization of proteolytic enzymes, such as trypsine, on polymer coated surfaces via photochemistry, for performing biochemical reactions. The trypsine was covalently attached onto an inner surface of a fused silica capillary (Polymicro Technologies, USA). The employed fused silica capillary was UV-light transparent, with inner diameter of 100 μm and outer diameter of 365 μm. A 10 cm long capillary (total volume of 0.785 μl) was connected to a 1 ml Hamilton glass syringe with a teflon tubing. The capillary was washed with 0.2 M sodium hydroxide during 30 minutes, then with Mili Q water (Millipore, France) for 10 min, 0.2 M hydrochloric acid for 30 min and finally with Mili Q water again during 10 min. All the washes were performed at a flow rate of 10 μl/min with the aid of a syringe pump (kd Scientific, USA). Subsequently, the capillary was flushed with nitrogen and dried in an oven at 80° C. during 1 hour. The capillary was washed with ethanol during 10 min and, subsequently, with trichloroethylene also for 10 min (flow rate of 10 μl/min). A 10% solution of γ-Methacryloxypropyl-trimethoxysilane (Sigma-Aldrich, France) in trichloroethylene was flowed into the capillary for 3 hours at a flow rate of 2 μl/min, then the capillary was washed with pure trichloroethylene for 30 min at 10 μl/min, flushed with Nitrogen and dried in an oven at 110° C. for 3 hours. A 5% solution (10 ml) of N-[Tris-(hydroxymethyl)-methyl]acrylamide (Sigma-Aldrich) in Mili Q water was degassed by bubbling with Argon for 3 hours. Ammonium persulfate and TEMED (N,N,N′,N′-Tetramethylethylene diamine, Sigma-Aldrich) were added to the monomer solution so their final concentrations were 1 mg/ml (ammonium persulfate) and 5 mg/ml (TEMED), respectively. The solution was homogenized and injected immediately into the silanized capillary. The solution was pushed into the capillary during 3 hours. In about 30 min, the viscosity of the solution started to increase, indicating the formation of polymer chains in the solution. After 3 hours of polymerization, the capillary was washed with Mili Q water, flushed with Nitrogen and stored at 4° C.

A solution of Trypsin (1.25 mg/ml) and water soluble derivative of benzophenone ((4-Benzoylbenzyl)trimethylammonium chloride) (12.5 mM), in 50 mM sodium phosphate buffer (pH 7.5) was prepared. This solution was pushed into the modified capillary at a flow rate of 5 μl/min. The capillary was placed about 7 cm away from a UV lamp (VL-215 L, 2×15 W, 365 nM Tube, Fisher Bioblock Scientific) and irradiated by UV-light (365 nm) during 30 min. The capillary was then washed with 50 mM phosphate buffer that contained 50 mM sodium chloride and 0.05% of Tween during 1 hour at a flow rate of 50 μl/min and, subsequently, it was washed with 50 mM sodium phosphate buffer during 2 hours at the same flow rate. The capillary was washed/equilibrated with 25 ammonium carbonate buffer (pH 7.8) overnight (at a flow rate of 0.2 μl/min) before it was used.

Solutions of Cytochrome C (concentrations of 2 μM (2 nM/μl) and/or 10 μM (10 nM/μl)) in 25 mM ammonium carbonate (pH 7.8) were pushed through the 10 cm, trypsine coated capillary (total volume of 0.785 μl) at flow rates of 0.08 μl/min (Péclet number ˜40) and/or 0.16 μl/min (Pëclet number ˜80). For each analysis, 15 pt of the Trypsine solution that passed through the capillary was collected at the capillary outlet. The samples were dried in a speed-vac, re-dissolved in 15 μl of 50% acetonitrile with 0.1% of formic acid and analyzed by MALDI/TOF (matrix assisted laser de-sorption ionization, time-of-flight mass spectrometry) (FIGS. 9A, 9B, 9C). It is worth noting that we have not seen intact Cytochrome C in any of the samples that were analyzed, suggesting that the digestion yield was close to 100%.

### Example 3

### Detection of Oligonucleotides in Solution

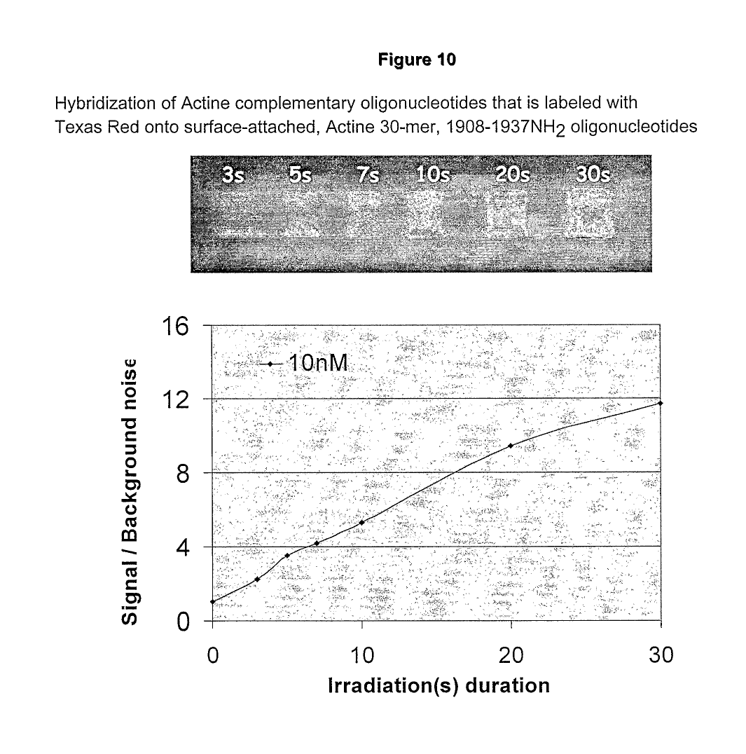

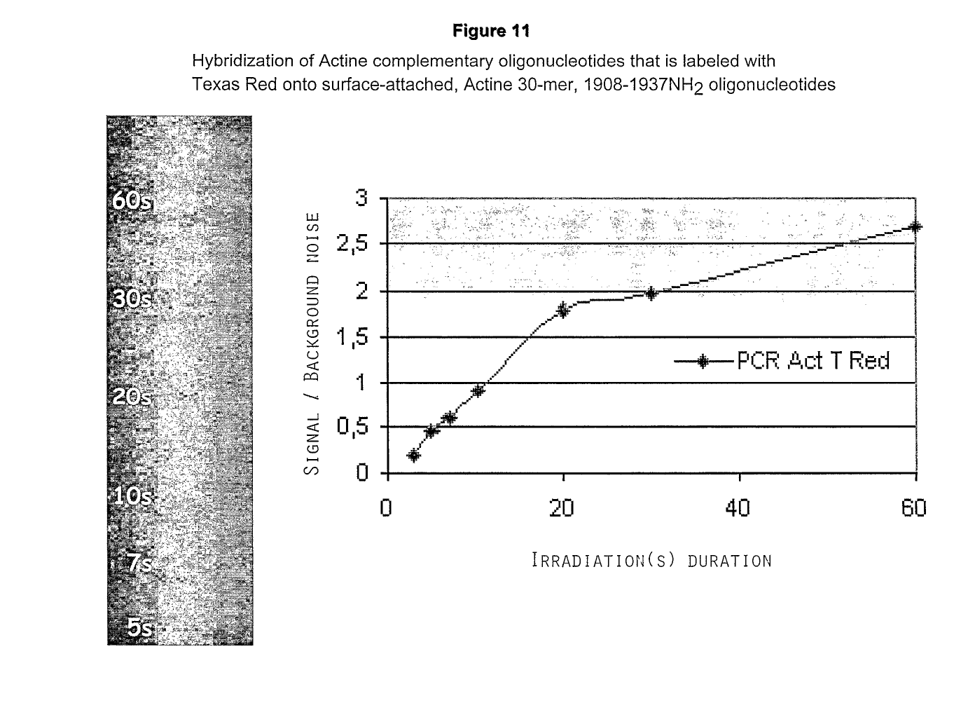

This example, with reference to FIG. 10, discusses one embodiment of the present invention and is directed to the immobilization of oligonucleotides on polymer coated surfaces via photochemistry, for detecting complementary oligonucleotides in solutions. The modification of a fused silica capillary with hydrophilic polymers, such as poly{N-[Tris-(hydroxymethyl)-methyl]acrylamide} was done according to the protocol described in the example 2.

For immobilization of oligonucleotides on the poly{N—[Tris-(hydroxymethyl)-methyl]acrylamide} surfaces, we prepared a solution containing an oligonucleotide (Actine_{—}30-mer, 1908-1037NH₂, with the NH₂ group on 5′ terminus) and water soluble derivate of benzophenone ((4-Benzoylbenzyl)trimethylammonium chloride). The final concentrations of the oligonucleotide and the water soluble derivative of benzophenone were 200 μM and 100 mM, respectively. The solution was injected into the polymer coated capillary. The capillary was positioned onto an inversed microscope (Olympus, Japan) and irradiated by a UV-light (365 nm) through a square mask (150×150 μm). The irradiation time was between 3 and 30 seconds (see FIG. 10). Each irradiation time corresponds to a different spot on the capillary. The light was focused onto the square mask by an objective (5×) and the power of the UV-light was 4 mW/cm². After the irradiation, the capillary was connected to a 1 ml Hamilton syringe with a Teflon tubing and washed (with the aid of a syringe pump (kd Scientific)) with 2 ml of a washing buffer (2×SSC+0.1% SDS) at a flow rate of 100 μl/min. The oligonucleotides attached onto the polymer coated surface were hybridized with a complementary oligonucleotide to the Actine oligonucleotide, that was labeled with Texas Red. The concentration of the complementary oligonucleotide was 10 nM. The oligonucleotide was dissolved in a phosphate buffer (1×PBS) containing 0.5 M sodium chloride, 10 mM ethylenediaminetetraacetic acid (EDTA), and 100 μg/ml of Salmon Sperm DNA (pH 8).

The solution of the complementary oligonucleotide was pushed in and out (forward/backward) of the capillary in an oscillatory manner during 20 minutes, at a flow rate of 9 μl/ml. After the hybridization was completed, the capillary was washed with 2 ml of a washing buffer (2×SSC+0.1% SDS; flow rate−100 μl/min). The fluorescent signal inside the capillary was measured on a Genetaq GS4 (Perkin Elmer, USA) scanner, at 615 nm and 42% gain.

### Example 4

### Detection of PCR Amplicon in Solution

This example, with reference to FIG. 11, discusses one embodiment of the present invention and is directed to immobilization of oligonucleotides on polymer coated surfaces via photochemistry, for detecting PCR amplicons in solutions. The modification of a fused silica capillary with hydrophilic polymers, such as poly{N-[Tris-(hydroxymethyl)-methyl]acrylamide} was prepared according to the protocol described in the example 2.

For immobilization of oligonucleotides on the poly{N-[Tris-(hydroxymethyl)-methyl]acrylamide}surfaces, we prepared a solution containing an oligonucleotide (Actine_{—}50-mer, 1908-1057NH₂, with the NH₂ group on 5′ terminus) and water soluble derivate of benzophenone ((4-Benzoylbenzyl)-trimethylammonium chloride). The final concentrations of the oligonucleotide and the water soluble derivative of benzophenone were 200 μM and 100 mM, respectively. The solution was injected into the polymer coated capillary. The capillary was positioned onto an inversed microscope (Olympus, Japan) and irradiated by a UV-light (365 nm) through a square mask (150×150 μm). The irradiation time was between 3 and 60 seconds (see FIG. 11). Each irradiation time corresponds to a different spot on the capillary. The light was focused onto the square mask by an objective (5×) and the power of the UV-light was 4 mW/cm². After the irradiation, the capillary was connected to a 1 ml Hamilton syringe with a Teflon tubing and washed (with the aid of a syringe pump (kd Scientific, USA)) with 2 ml of a washing buffer (2×SSC and 0.1% SDS) at a flow rate of 100 μl/min. The oligonucleotides attached onto the polymer coated surface were pre-hybridized with pre-hybridization buffer (20 mM phosphate buffer, 1M NaCl, 5.2 mM KCl 0.1% Tween, 2×Denhardt and 20 μg/ml of Salmon Sperm DNA). The pre-hybridization was accomplished in an oscillatory manner (forward/backward), at 100 μl/min during 5 minutes. After the pre-hybridization step, the oligonucleotides attached to the capillary inner surface were hybridized with a PCR (Actine) amplicon. The PCR amplicon was not purified and it was dissolved in a PCR hybridization buffer (1×PBS, 0.5 M NaCl, 20 mM EDTA, 100 μg/ml of Salmon Sperm DNA). The length of the amplicon was 680-bp and the amplified DNA was labeled with Texas Red fluorescent dye. The PCR amplicon was denatured at 95° C. during 5 minutes prior the hybridization step. The hybridization of the PCR amplicon with the oligonucleotides grafted on the capillary surface was done during 20 minutes, at 42° C. in an oscillatory manner (forward/backward) at a flow rate of 8 μl/min. After the hybridization, the capillary was washed with 2 ml of the washing buffer (2×SSC+0.1% SDS) at a flow rate of 100 μl/min. The fluorescent signal inside the capillary was measured on a Genetaq GS4 (Perkin Elmer, USA) scanner, at 615 nm and 56% gain (FIG. 11).

1: A method for the grafting of a molecule to at least one predefined region of a solid substrate, wherein the solid substrate comprises a layer of a linker that has a resistance to the adsorption of the molecule, said method comprising the steps of:
contacting the solid substrate with a solution wherein the molecule to be grafted and a photo-sensitizer are solubilized; and
photo-irradiating the predefined region of the solid substrate.

2. The method according to claim 1, wherein the predefined region of the solid substrate is irradiated by using a mask.

3. The method according to claim 1, wherein the molecule is selected from the group consisting of proteins, nucleic acids and their analogs, sugars, lipids, steroids, enzymes, peptides, and glycoproteins,

4. The method according to claim 1, wherein the substrate is selected from the group consisting of glass, silicon, fused silica, polymers, metals, metal oxides and ceramics.

5. The method according to claim 1, wherein the substrate is selected from the group consisting of planar surfaces, beads, microtiter plates, and capillaries.

6. The method according to claim 1, wherein the substrate is at least one fused silica capillary with UV transparent coating.

7. The method according to claim 1, wherein the layer of a linker is selected from the group consisting of synthetic hydrophilic poly(oligo)mer and natural poly(oligo)mer.

8. The method according to claim 7, wherein the linker is a polymer containing amino or H-donor groups.

9. The method according to claim 7, wherein the linker is selected from polymer brushes synthesized from acrylamide, N-(3-aminopropyl) methacrylamide, N-[(3-dimethylamino) propyl]methacrylamide, N-[tris-(hydroxymethyl)-methyl]acrylamide, polyethylene glycols (PEG), polyethylene oxides (PEO), copolymers of PEG with polypropylene glycols (PPG), copolymers of PEO with polypropylene glycols (PPG), copolymers of PEG with polypropylene oxides (PPO), copolymers of PEO with polypropylene oxides (PPO), copolymers of PEG with polydimethylsiloxanes (PDMS), copolymers of PEO with polydimethylsiloxanes (PDMS), copolymers of PEG with derivatives of polyacrylamides, or copolymers of PEO with derivatives of polyacrylamides.

10. The method according to claim 2, wherein the photo-sensitizer is at least one carbonyl-containing photo-sensitizer.

11. The method according to claim 10, wherein the photo-sensitizer is selected from the group consisting of benzophenone, anthraquinone, camphorquinone, thioxanthone derivatives, benzophenone iso(thio)cyanate, substituted benzoylbenzoic acids, benzoylbenzylbromide, (benzoylbenzyl)-trimethylammonium chloride, anthraquinone, anthraquinone-containing carboxylic acids, bromomethyl-anthraquinone, anthraquinone sulfonic acid, and camphorquinone-10-sulfonic acid.

12. The method according to claim 1, wherein the photo-sensitizer is covalently or non-covalently attached to the molecule.

13. The method according to claim 1, wherein a tertiary amine is solubilized in the solution of the photo-sensitizer and the molecule or bound to the solid substrate.

14. The method 3 according to claim 1, wherein irradiation is provided by a mercury arc lamp, a laser, a CRT, a LED, Resonant Microcavity Anodes, photodiodes, or broad wavelength lamps.

15. The method according to claim 1 further comprising the step of washing the substrate's surface.

16. The method according to claim 1 further comprising the steps of:
washing the substrate's surface;
contacting the solid substrate with a solution wherein a second molecule to be grafted and a photo-sensitizer are solubilized- and
photo-irradiating another region of the solid substrate.

17. The method for the detection of multiple interaction events of one analyte in parallel with at least two molecules, said method comprising a method according to claim 1.

18. The method according to claim 17, further comprising a detection step based on at least one detection method selected from the group consisting of fluorescence, luminescence, radioactivity, SPR, and mass spectrometry.

19. The method for the detection of multiple interaction events of one analyte with at least two molecules, said method comprising:
using a fused silica capillary,
pre-cleaning of the inner capillary surface,
chemically modifying the inner capillary surface with a silane derivative,
grafting the at least two molecules using a method according to claim 1,
filling the capillary with the analyte sample, and
detecting interactions with the surface-bound molecules.
